(19) United States
(12) Patent Application Publication
(10) Pub. No.: US 2008/0039533 A1
(43) Pub. Date: Feb. 14, 2008

(54) Bioactive Compositions Comprising Triazines

(75) Inventors: Hassan Sahouani, Hastings, MN (US); Robert A. Scherrer, White Bear Lake, MN (US); Mouhannad Jumaa, Foster City, CA (US); Isidro A. E. Zarraga, St. Paul, MN (US); Kim M. Vogel, Lake Elmo, MN (US); Dennis E. Vogel, Lake Elmo, MN (US); Wei Zou, Woodbury, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, ST. PAUL, MN (US)

(21) Appl. No.: 10/595,050

(22) PCT Filed: Jul. 29, 2004
(86) PCT No.: PCT/US04/24515

§ 371 Date: Jan. 18, 2006

Related U.S. Application Data
(60) Provisional application No. 60/491,638, filed on Jul. 31, 2003.
(60) Provisional application No. 60/491,631, filed on Jul. 31, 2003.

(51) Int. Cl. A61K 47/22

(52) U.S. Cl. 514/784

(57) ABSTRACT

Compositions and methods including a bioactive compound and a triazine compound comprising: formula (I) or formula (II) and proton tautomers and salts thereof . Each R₂ is independently selected from any electron donating group, electron withdrawing group and electron neutral group. R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃.

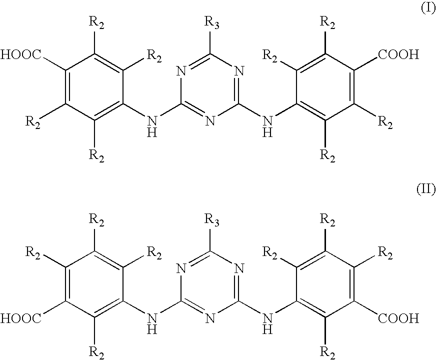

### FIELD

The present invention relates to bioactive compositions comprising a bioactive compound and a triazine compound. In particular, the present invention relates to pharmaceutical compositions comprising a drug.

### BACKGROUND

The delivery of a bioactive compound to a living organism is generally affected by a number of parameters beyond the actual chemical identity and pharmacological activity of the bioactive compound. Formulation additives other than the bioactive compound are commonly used to alter the physicochemical properties of a product having bioactive function. As an example, pharmaceutical dosage forms (i.e., dosages containing a drug or active pharmaceutical ingredient) typically contain one or more non-pharmaceutically active ingredients that are referred to as excipients. There are a wide variety of purposes for excipients, just a few examples of which are adjusting the physical form of a dosage (e.g., tablet formation, viscosity adjustment in semi-solids), aiding in drug solubilization or stabilization, or enhancing the uptake of drug in a living organism (e.g., permeation enhancement, selective site targeting).

### SUMMARY OF THE INVENTION

The present invention provides, among other things, a bioactive composition comprising a bioactive compound and a triazine compound comprising: 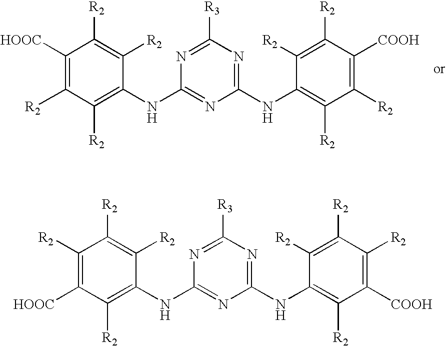
and proton tautomers and salts thereof. Each R₂ is independently selected from any electron donating group, electron withdrawing group and electron neutral group. R₃ is selected from the group consisting of: substituted heteroaromatic rings, unsubstituted heteroaromatic rings, substituted heterocyclic rings, and unsubstituted heterocyclic rings, that are linked to the triazine group through a nitrogen atom within a ring of R₃.

Another aspect of the invention includes a method for increasing the solubility of a bioactive compound in a bioactive composition comprising providing a bioactive compound, providing a triazine compound comprising: 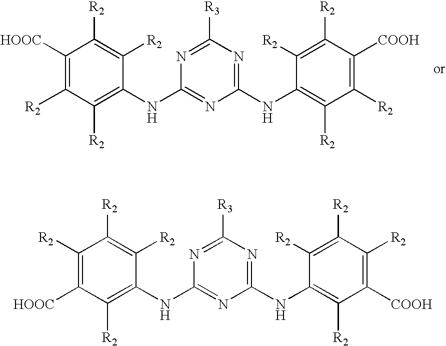
and proton tautomers and salts thereof. The bioactive compound, the triazine compound, and a solvent are combined to form a composition characterized in that the amount of dissolved bioactive compound in the composition is greater than the amount of bioactive compound dissolvable in the same composition not containing the triazine compound. In other words, the triazine can be used to increase the amount of bioactive compound that can be dissolved in a composition The triazine compound is characterized in that each R₂ is independently selected from any electron donating group, electron withdrawing group and electron neutral group. R₃ is selected from the group consisting of: substituted heteroaromatic rings, unsubstituted heteroaromatic rings, substituted heterocyclic rings, and unsubstituted heterocyclic rings, that are linked to the triazine group through a nitrogen atom within a ring of R₃.

In still another aspect, the present invention includes a method for increasing the stability of a bioactive compound in a bioactive composition comprising providing a bioactive compound, and providing a triazine compound comprising: 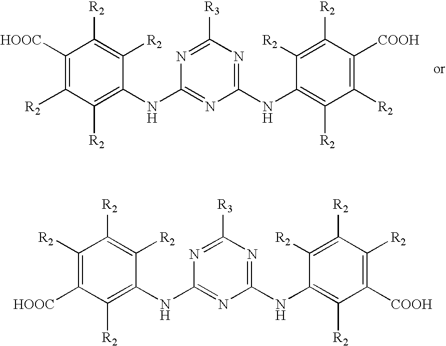
and proton tautomers and salts thereof. The bioactive compound, the triazine compound, and a solvent are combined to form a bioactive composition characterized in that the stability of the bioactive compound in the composition is greater than the stability of the bioactive compound in the same composition not containing the triazine compound. In other words, the triazine compound can be used to stabilize the bioactive compound. The triazine compound is characterized in that each R₂ is independently selected from any electron donating group, electron withdrawing group and electron neutral group. R₃ is selected from the group consisting of: substituted heteroaromatic rings, unsubstituted heteroaromatic rings, substituted heterocyclic rings, and unsubstituted heterocyclic rings, that are linked to the triazine group through a nitrogen atom within a ring of R₃.

These and other features and advantages of the invention are described below in connection with various illustrative embodiments of the invention.

### DETAILED DESCRIPTION

The present invention provides a composition comprising a bioactive compound and a triazine compound comprising: 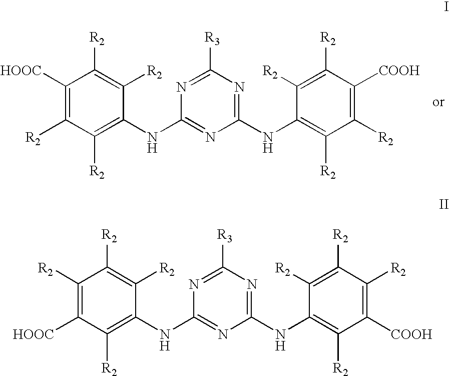
and proton tautomers and salts thereof. Each R₂ is independently selected from any electron donating group, electron withdrawing group and electron neutral group. R₃ is selected from the group consisting of substituted and unsubstituted heteroaromatic rings linked to the triazine group through a nitrogen atom within a ring of R₃.

Formula I above shows an orientation of the carboxy (—COOH) group which is para with respect to the amino linkage to the triazine backbone of the compound. The carboxy group may also be meta with respect to the amino linkage, as shown in formula II. It should also be understood that the two positions may be mixed, such that one carboxy group is para and the other is meta.

Each R₂ is independently selected from any electron donating group, electron withdrawing group and electron neutral group. Preferably, R₂ is hydrogen or a substituted or unsubstituted alkyl group. More preferably, R₂ is hydrogen, an unsubstituted alkyl group, or an alkyl group substituted with a hydroxy, ether, ester, sulfonate, or halide functional group. Most preferably R₂ is hydrogen.

R₃ may be selected from the group consisting of: substituted heteroaromatic rings, unsubstituted heteroaromatic rings, substituted heterocyclic rings, and unsubstituted heterocyclic rings, that are linked to the triazine group through a nitrogen atom within a ring of R₃. R₃ can be, but is not limited to, heteroaromatic rings derived from pyridine, pyridazine, pyrimidine, pyrazine, imidazole, oxazole, isoxazole, thiazole, oxadiazole, thiadiazole, pyrazole, triazole, triazine, quinoline, and isoquinoline. Preferably R₃ comprises a heteroaromatic ring derived from pyridine or imidazole. A substituent for the heteroaromatic ring R₃ may be selected from, but is not limited to, any of the following substituted and unsubstituted groups: alkyl, carboxy, amino, alkoxy, thio, cyano, amide, sulfonate, hydroxy, halide, perfluoroalkyl, aryl, ether, and ester. The substituent for R₃ is preferably selected from alkyl, sulfonate, carboxy, halide, perfluoroalkyl, aryl, ether, and alkyl substituted with hydroxy, sulfonate, carboxy, halide, perfluoroalkyl, aryl, and ether. When R₃ is a substituted pyridine the substituent is often located at the 4-position. When R₃ is a substituted imidazole the substituent is often located at the 3-position. Suitable examples of R₃ include, but are not limited to: 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl, shown in formulae IV to XIII below. Examples of heterocyclic rings that R₃ may be selected from include, for example, morpholine, pyrrolidine, piperidine, and piperazine.

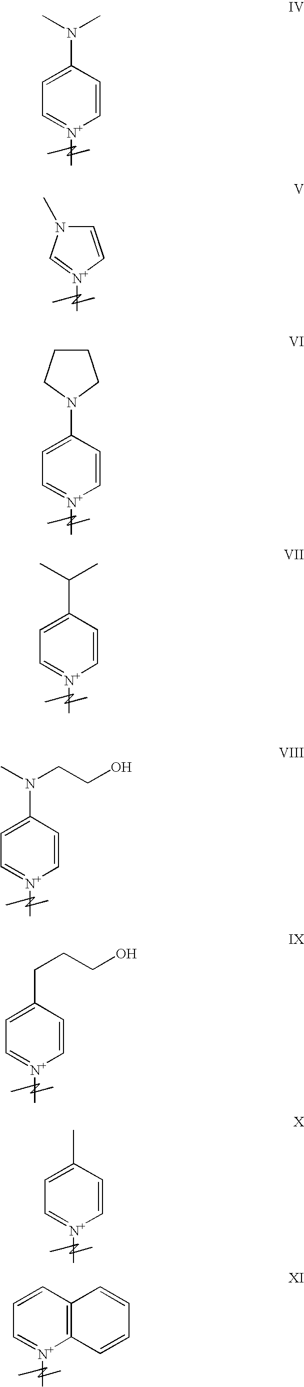

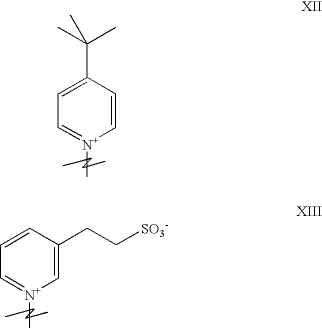

The R₃ group shown in formula V above may also have a substituent group other than methyl attached to the imidazole ring, as shown below, 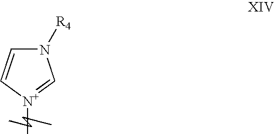
where R₄ is hydrogen or a substituted or unsubstituted alkyl group. In some instances R₄ is hydrogen, an unsubstituted alkyl group, or an alkyl group substituted with a hydroxy, ether, ester, sulfonate, or halide functional group. For example, R₄ may be propyl sulfonic acid, methyl, or oleyl.

As depicted above the triazine of formula I is neutral, however triazine molecules of the present invention may exist in an ionic form wherein they contain at least one formal positive charge. In a preferred embodiment, the triazine molecule may be zwitterionic. An example of such a zwitterionic triazine molecule, 4-{[4-(4-carboxyanilino)-6-(1-pyridiniumyl)-1,3,5-triazin-2-yl]amino}benzoate, is shown in formula III below where R₃ is a pyridine ring linked to the triazine group through the nitrogen atom of the pyridine ring. The pyridine nitrogen carries a positive charge and one of the carboxy functional groups carries a negative charge (and has a dissociated cation, such as a hydrogen atom), —COO⁻.

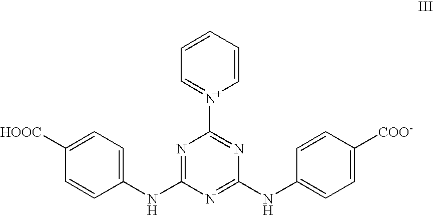

The molecule shown in formula III may also exist in other tautomeric forms, such as where both carboxy functional groups carry a negative charge and where positive charges are carried by one of the nitrogens in the triazine group and the nitrogen in the pyridine group.

As described in U.S. Pat. No. 5,948,487 (Sahouani, et al.), triazine derivatives with formula I may be prepared as aqueous solutions, or may be prepared as salts which can later be re-dissolved to form an aqueous solution. A typical synthetic route for the triazine molecules shown in I above involves a two step process. Cyanuric chloride is treated with 4-aminobenzoic acid to give 4-{[4-(4-carboxyanilino)-6-chloro-1,3,5-triazin-2-yl]amino}benzoic acid. This intermediate is treated with a substituted or unsubstituted nitrogen-containing heterocycle. The nitrogen atom of the heterocycle undergoes nucleophilic displacement of the chlorine atom on the triazine to form the corresponding chloride salt. The zwitterionic derivative, such as that shown in formula III above, is prepared by dissolving the chloride salt in ammonium hydroxide and passing it down an anion exchange column to replace the chloride with hydroxide, followed by solvent removal. Alternative structures, such as that shown in II above, may be obtained by using 3-aminobenzoic acid instead of 4-aminobenzoic acid.

In one embodiment, the triazine contains at least one formal positive charge. The triazine may also be zwitterionic, that is, carrying at least one formal positive and one formal negative charge. Zwitterionic triazines of the present invention will carry at least one negative charge through a carboxy group having a dissociated hydrogen atom, —COO⁻. The negative charge may be shared among the multiple carboxy functional groups present, such that a proper representation of the triazine consists of two or more resonance structures. Alternatively, the negative or partial negative charges may be carried by other acid sensitive groups in the triazine.

In one aspect, the triazine can be used to form a chromonic phase or assembly when in an aqueous solution. Chromonic phases or assemblies are well known (see, for example, Handbook of Liquid Crystals, Volume 2B, Chapter XVIII, Chromonics, John Lydon, pp. 981-1007, 1998) and consist of stacks of flat, multi-ring aromatic molecules. The molecules typically consist of a hydrophobic core surrounded by hydrophilic groups. The stacking takes on a number of morphologies, but is typically characterized by a tendency to form columns created by a stack of layers. Ordered stacks of molecules can be formed that grow with increasing concentration, but they are distinct from micellar phases in that they generally do not have surfactant-like properties and do not exhibit a critical micellar concentration. Typically, the chromonic phases will exhibit isodesmic behavior, that is, addition of molecules to the ordered stack leads to a monotonic decrease in free energy. In some embodiments, the triazines will form either a chromonic M or N phase in aqueous solution. The chromonic M phase typically is characterized by ordered stacks of molecules arranged in a hexagonal lattice. The chromonic N phase is characterized by a nematic array of columns, that is, there is long range ordering along the columns characteristic of a nematic phase, but there is little or no ordering amongst the columns, thus it is less ordered than the M phase. The chromonic N phase typically exhibits a schlieren texture, which is characterized by regions of varying index of refraction in a transparent medium.

Although not wishing to be bound by any particular theory, it is believed that the ordered chromonic phase can contribute to increased solubility of a bioactive compound by providing sites within the ordered stacks where the bioactive compounds may reside and where they will have little interaction with the bulk solvent, such as the aqueous phase, where the bioactive compounds may have lower solubility. Similarly, the chromonic ordered phase may be able to isolate the bioactive compounds from the solvent and potentially from each other, since the bioactive compounds may be interleaved or intercalated on a molecular scale between the triazine molecules. Thus, bioactive compounds that are unstable in the presence of other chemical components of the composition, for example, bulk solvent, other excipients, and low-level impurities, may be protected from degradation by the chromonic phase. Bioactive compounds that are unstable in the presence of other physical or packaging components of the dosage form, for example, walls of a syringe or vial, metered dose inhaler canisters, may be protected from degradation by the chromonic phase.

In some embodiments, compositions of the present invention may comprise a surfactant. Suitable surfactants include, for example, long chain saturated fatty acids or alcohols and mono or poly-unsaturated fatty acids or alcohols. Oleyl phosphonic acid is a preferred surfactant. Although not wishing to be bound by any particular theory, it is thought that the surfactant aids in dispersing the bioactive compound.

Some compositions of the present invention may comprise an alkaline compound. Examples of suitable alkaline compounds include ethanolamine, sodium or lithium hydroxide, or amines such as mono, di, triamines or polyamines. Again, although not wishing to be bound by any particular theory, it is thought that alkaline compounds aid in dissolving the triazine compound.

A bioactive compound as used herein is defined as a compound intended for use in the diagnosis, cure, mitigation, treatment or prevention of disease, or to affect the structure or function of a living organism. Examples of bioactive compounds include drugs, herbicides, pesticides, pheromones, and antifingal agents. Drugs (i.e., pharmaceutically active ingredients) are bioactive compounds of particular interest. Alternatively, herbicides and pesticides are examples of bioactive compounds intended to have a negative effect on a living organism, such as a plant or pest. Although any type of drug may be employed with compositions of the present invention, of particular interest are drugs that are relatively unstable when formulated as solution, suspension, or semi-solid dosage forms, and those that have poor solubility in conventional carriers. Examples of suitable drugs include antiinflammatory drugs, both steroidal (e.g., hydrocortisone, prednisolone, triamcinolone) and nonsteroidal (e.g., naproxen, piroxicam); systemic antibacterials (e.g., erythromycin, tetracycline, gentamycin, sulfathiazole, nitrofurantoin, vancomycin, penicillins such as penicillin V, cephalosporins such as cephalexin, and quinolones such as norfloxacin, flumequine, ciprofloxacin, and ibafloxacin); antiprotazoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors, and angiotensin converting enzyme inhibitors (e.g., captopril, lisinopril); other antihypertensives (e.g., propranolol); leukotriene antagonists; anti-ulceratives such as H2 antagonists; steroidal hormones (e.g., progesterone, testosterone, estradiol); local anesthetics (e.g., lidocaine, benzocaine, propofol); cardiotonics (e.g., digitalis, digoxin); antitussives (e.g., codeine, dextromethorphan); antihistamines (e.g., diphenhydramine, chlorpheniramine, terfenadine); immune response modifiers (e.g., imiquimod, resiquimod); narcotic analgesics (e.g., morphine, fentanyl); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatripan); antiarrhythmic agents (e.g., flecainide); antiemetics (e.g., metaclopromide, ondansetron); anticancer agents (e.g., methotrexate); neurologic agents such as anti-depressants (e.g., fluoxetine) and anti-anxiolytic drugs (e.g., paroxetine); hemostatics; and the like, as well as pharmaceutically acceptable salts and esters thereof. Proteins and peptides are particularly suitable for use with compositions of the present invention, as are monoclonal antibodies. Drugs that are poorly soluble in aqueous solutions or that degrade in aqueous environments are particularly applicable for use with compositions of the present invention. The amount of drug that constitutes a therapeutically effective amount can be readily determined by those skilled in the art with due consideration of the particular drug, the particular carrier, the particular dosing regimen, and the desired therapeutic effect.

The weight ratio of drug to the triazine compound will typically be greater than about 1:1000, usually greater than about 1:100, often greater than about 1:20, and sometimes greater than about 1:10. The weight ratio of drug to the triazine compound will typically be less than about 10:1, usually less than about 1.5:1, often less than about 1:1, and sometimes less than about 1:2.

The triazine compound is generally itself non-therapeutic. The triazine compound may alter the dosage form and may influence, for example, the amount of drug delivered to a site in a living organism in a bioavailable form, which can clearly affect the therapeutic activity of the drug. Although this affect on therapeutic activity is a direct result of the function of the triazine compound in the present invention, it is normally preferred that the triazine compound itself is non-therapeutic once it is dissociated from the drug. Thus, by non-therapeutic it is meant that the triazine compound has no appreciable therapeutic activity when delivered to an organism, e.g., such as an animal, in the form of isolated molecules. The triazine compound is generally largely inert with relation to biological interactions with an organism and will thus serve only as a carrier for the drug. The triazine compound is preferably non-toxic, non-mutagenic, and non-irritating.

Compositions of the present invention may find use in a variety of routes of drug delivery, including oral, such as tablets, capsules, liquid solutions, and syrups; by intravenous, intramuscular, or intraperitoneal injection, such as aqueous or oil solutions or suspensions; by subcutaneous injection; or by incorporation into transdermal, topical, or mucosal dosage forms, such as creams, gels, adhesive patches, suppositories, and nasal sprays. Compositions of the present invention may also be implanted or injected into various internal organs and tissues, for example, cancerous tumors, or may be directly applied to internal body cavities, such as during surgical procedures. Compositions of the present invention may also be suitable for use in inhalation dosage forms, such as pressurized meter dose inhalers, for example, those described in U.S. Pat. No. 5,836,299 (Kwon, et al.), the disclosure of which is incorporated by reference; and nebulizers, for example, those described in U.S. Pat. No. 6,338,443 (Piper, et al.), the disclosure of which is incorporated by reference. In one type of embodiment a liquid or semi-solid composition of the present invention may be contained within a capsule for oral delivery that is designed to release the composition at a specific location within the gastrointestinal tract. In another type of embodiment, the composition of the present invention may be the discontinuous phase of a water-in-oil emulsion.

Compositions of the present invention can optionally include one or more other ingredients in addition to the bioactive compound and the triazine compound, such as, for example, initiators, fillers, plasticizers, cross-linkers, tackifiers, binders, antioxidants, stabilizers, surfactants, solubilizers, buffers, permeation enhancers, adhesives, viscosity enhancing agents, coloring agents, flavoring agents, and mixtures thereof. A combination of bioactive compounds may also be used.

In another aspect, the present invention comprises a method for preparing a bioactive composition comprising provision of a bioactive compound and provision of a triazine compound comprising a molecule of formula I or II, wherein each R₂ is independently selected from any electron donating group, electron withdrawing group and electron neutral group and R₃ is selected from the group consisting of substituted and unsubstituted heteroaromatic rings linked to the triazine group through a nitrogen atom within a ring of R₃, and proton tautomers and salts thereof. The bioactive compound, the triazine compound, and a solvent are combined to form a bioactive composition. The solvent is a liquid or semi-solid capable of dissolving or dispersing the bioactive compound and the triazine compound. The solvent may remain in the final dosage form. In a pharmaceutical composition, for example, a pharmaceutically acceptable excipient, such as water, ethanol, propylene glycol, or 1,1,1,2-tetrafluoroethane, may remain in the final dosage form. Alternatively, the solvent may be used for processing purposes and be removed prior to preparation of a final dosage form. Process solvents may be removed by any process known to one of skill in the art, including for example, distillation or solvent stripping, air impingement drying, air drying or evaporation, and/or vacuum drying. Typical process solvents include, for example, methanol, ethyl acetate, heptane, hexane, and acetone. Solvents that are acceptable for use in the final dosage form, such as water, may also be used as process solvents.

Compositions of the present invention may be prepared by mixing triazines with a bioactive compound. For example, the triazine may be dissolved in an aqueous solution and the bioactive compound is added to the triazine solution. It may be desirable to prepare a concentrated stock solution of triazine and bioactive compound that is subsequently diluted to prepare a final dosage form. Likewise, additional ingredients may be added to the initial triazine solution or be added to the resulting mixtures of triazine and bioactive. In a preferred embodiment, the triazine solution exhibits a chromonic M or N phase. This chromonic solution may be moderately or highly viscous. Typical solution viscosities for a chromonic solution containing 15% by weight triazine will be between about 100 and about 700 centipoise at room temperature, and more preferably between about 200 and 400 centipoise at room temperature. It may be desirable to heat one or more of the intermediate solutions to assist in dissolution or mixing of one or more of the ingredients of the final dosage form.

In another example, the bioactive compound may be dissolved in an aqueous solution and the triazine is added to the bioactive compound solution.

In one aspect, the present invention can be used as a method for increasing the solubility of a bioactive compound in a bioactive composition comprising provision of a bioactive compound and provision of a triazine compound comprising a molecule of formula I or II, wherein each R₂ is independently selected from any electron donating group, electron withdrawing group and electron neutral group and R₃ is selected from the group consisting of substituted and unsubstituted heteroaromatic rings linked to the triazine group through a nitrogen atom within a ring of R₃, and proton tautomers and salts thereof. The bioactive compound, the triazine compound, and a solvent are combined to form a bioactive composition characterized in that the amount of dissolved bioactive compound in the composition is greater than the amount of dissolved bioactive compound in the same composition not containing the triazine compound. The ratio of the amount of bioactive compound dissolvable in a composition using triazine compound to the amount of bioactive compound dissolvable in the same composition not containing the triazine compound can be greater than about 1.5:1 and in some instances greater than 2:1. In some embodiments the ratio of the amount of bioactive compound dissolvable in the composition using triazine compound to the amount of bioactive compound dissolvable in the same composition not containing the triazine compound may be greater than about 100:1.

In another aspect, the present invention comprises a method for increasing the stability of a bioactive compound in a bioactive composition by proving a bioactive compound and a triazine compound comprising a molecule of formula I or II, wherein each R₂ is independently selected from any electron donating group, electron withdrawing group and electron neutral group and R₃ is selected from the group consisting of substituted and unsubstituted heteroaromatic rings linked to the triazine group through a nitrogen atom within a ring of R₃, and proton tautomers and salts thereof. The bioactive compound, the triazine compound, and a solvent are combined to form a bioactive composition characterized in that the stability of the bioactive compound in the composition is greater than the stability of the bioactive compound in the same composition not containing the triazine compound. Stability may be affected by storage conditions, such as temperature, relative humidity (RH), and the like. Stability of bioactive compositions of the present invention is typically increased and measured under typical storage conditions, such as 25° C./60% RH and 40° C./75% RH.

Stability is often characterized by measuring the reduction in the amount of bioactive compound in the composition as a function of time where the initial amount of bioactive compound is considered to be 100% content. For example, measurement of 95% of the initial amount of bioactive compound is equivalent to a reduction of 5% of the initial amount of bioactive compound. Dosage forms using or including the methods and compositions of the present invention may be characterized in that the reduction in amount of bioactive compound over time is less than the reduction in amount of bioactive compound over time in the same dosage form not containing the triazine compound. The lessened reduction in amount of bioactive compound is typically observed over lengths of time ranging from 4 weeks to 3 years, including for example, 1 month, 3 months, 6 months, 1 year, and 2 years. The ratio of the reduction in amount of bioactive compound over time compared to the reduction in amount of bioactive compound over time for a like dosage form not containing the triazine compound is preferably less than about 3:4, more preferably less than about 1:2, and most preferably less than about 1:4. The dosage form may comprise more than one bioactive compound, for instance, a combination of two bioactives, such as enalapril and felodipine, and an improvement in stability of such a dosage form may be seen in one or both of the bioactive compounds.

In another aspect, the present invention comprises a method for drug delivery comprising provision of a bioactive composition comprising a drug and a triazine compound comprising a molecule of formula I or II, wherein each R₂ is independently selected from any electron donating group, electron withdrawing group and electron neutral group and R₃ is selected from the group consisting of substituted and unsubstituted heteroaromatic rings linked to the triazine group through a nitrogen atom within a ring of R₃, and proton tautomers and salts thereof. The bioactive composition is delivered to an organism, and allowed to remain in contact with a portion of the organism for a period of time sufficient to provide a therapeutic effect resulting from delivery of the drug. The bioactive composition may be delivered to an animal, e.g., orally, by intravenous, subcutaneous, intratumoral, or intramuscular injection, oral or nasal inhalation, or any other suitable method for drug delivery known in the art.

### EXAMPLES

### Examples 1-4

Imiquimod solubility in basic solutions containing a triazine compound was determined as follows. A solution was prepared by adding approximately 1 g of 1-[4,6-bis(4-carboxyanilino)-1,3,5-triazin-2-yl]-4-(dimethylamino)pyridinium chloride to 9 g of distilled water containing a molar equivalent amount of a counterion base. The solution was heated to 70° C., an excess of imiquimod (approximately 0.1 g) was added to the solution, and stirred for approximately 14 hours. The solution was then allowed to cool to room temperature for at least 5 hours prior to filtering through a 5.0 μm filter to remove the undissolved solids. These solutions had a pH of between 9 and 10. Imiquimod concentration was then determined by HPLC, at which time the solution was further filtered through a 0.45 μm filter. The concentration of triazine compound in the prepared solution, the type of counterion base, and the measured imiquimod solubility are shown in Table 1 below. Imiquimod solubility in a buffer solution having a pH of 6.05 and not containing a triazine compound is 0.02 mg/mL. Imiquimod solubility in a buffer solution having a pH of 7.82 and not containing a triazine compound is 0.0012 mg/mL.

TABLE 1
Imiquimod solubility in solutions with triazine compounds

| | % triazine | | Imiquimod Solubility |
|---|---|---|---|
| Example | cmpd. | Counterion base | [% w/w] |
| 1 | 10 | ethanolammonium | 0.16 |
| 2 | 20 | ethanolammonium | 0.22 |
| 3 | 10 | isopropylammonium | 0.38 |
| 4 | 10 | Polyoxypropylene- | 1.23 |
| | | glycolammonium (D400) | |

### Examples 5-9

Lidocaine solubility in solutions containing a triazine compound was determined as follows. A stock solution was prepared by combining 1-[4,6-bis(4-carboxyanilino)-1,3,5-triazin-2-yl]-4-(dimethylamino)pyridinium chloride (6.0027 g), ethanolamine (1.35 g), and distilled water (18.00 g). This solution was stirred until the solids were dissolved to give a solution having 20% w/w triazine compound. Solutions having varying concentration of triazine (shown in Table 2) were prepared by removing an aliquot from the stock solution and diluting the aliquot with distilled water to reach each triazine concentration. An excess (at least 3-fold) of lidocaine was added to each of the solutions and shaken at ambient temperature for at least 24 hours.

The solutions were filtered through a 0.45 μm filter to remove the undissolved solids and then analyzed by HPLC for lidocaine concentration. The concentration of triazine compound in the prepared solution and the measured lidocaine solubility are shown in Table 2 below.

TABLE 2
Lidocaine solubility in solutions with triazine compounds

| | Concentration triazine cmpd. | Lidocaine Solubility |
|---|---|---|
| Example | [% w/w] | [% w/w] |
| 5 | 5 | 0.79 |
| 6 | 7.5 | 0.74 |
| 7 | 10 | 0.78 |
| 8 | 15 | 0.86 |
| 9 | 20 | 1.18 |

### Examples 10-14

Alendronate solubility in solutions containing a triazine compound was determined as follows. A stock solution was prepared by combining 1-[4,6-bis(4-carboxyanilino)-1,3,5-triazin-2-yl]-4-(dimethylamino)pyridinium chloride (4.02169 g), ethanolamine (0.8898 g), and distilled water (12.0019 g). This solution was stirred until the solids were dissolved to give a solution having 20% w/w triazine compound. Solutions having varying concentration of triazine (shown in Table 3) were prepared by removing an aliquot from the stock solution and diluting the aliquot with distilled water to reach the desired triazine concentration. An excess of alendronate was added to each of the solutions and shaken at ambient temperature for at least 24 hours.

The solutions were filtered through a 0.45 μm filter to remove the undissolved solids and then analyzed by capillary electrophoresis (Instrument: G1600AX^{3D}CE system from Agilent technologies; Capillary: 30 cm×50μid fused silica; Buffer: 20 mM pyridine dicarboxylic acid+200 μg/mL polybrene flow reversal agent, pH 12; Capillary prep: 3 minute buffer flush; Capillary temp: 25° C.; Injection: pressure injection of 10 sec at 50 mbar; Potential: −20 kV; Run time: 15 min; Detector: UV, 350 nm with reference at 230 nm) for alendronate concentration. The concentration of triazine compound and the measured alendronate solubility are shown in Table 3 below. The solubility of alendronate in distilled water was determined by adding an excess of alendronate to distilled water, shaking for 24 hours, filtering, and analyzing by capillary electrophoresis, as above. The solubility of alendronate in distilled water was 3.1% [w/w].

TABLE 3
Alendronate solubility in solutions with triazine compounds

| | | Alendronate Solubility |
|---|---|---|
| Example | % triazine cmpd. | [% w/w] |
| 10 | 5 | 7.2 |
| 11 | 7.5 | 8.1 |
| 12 | 10 | 11.0 |
| 13 | 15 | 13.5 |
| 14 | 20 | 11.2 |

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

1. A bioactive composition comprising:
a bioactive compound, and
a triazine compound comprising: 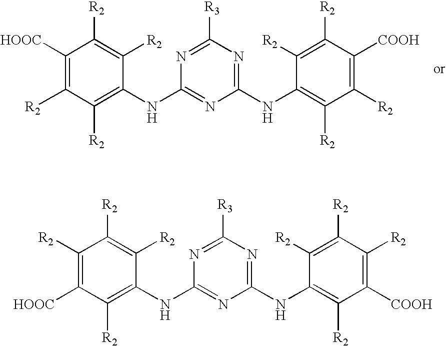
wherein each R₂ is independently selected from any electron donating group, electron withdrawing group and electron neutral group; and
R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃,
and proton tautomers and salts thereof.

2. A bioactive composition according to claim 1, wherein each R₂ is independently selected from the group consisting of hydrogen, an unsubstituted alkyl group, or an alkyl group substituted with a hydroxy, ether, ester, sulfonate, or halide functional group.

3. A bioactive composition according to claim 1, wherein R₃ comprises a heteroaromatic ring derived from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

4. A bioactive composition according to claim 3, wherein R₃ comprises a heteroaromatic ring derived from a pyridine or imidazole.

5. A bioactive composition according to claim 4, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

6. A bioactive composition according to claim 1 further comprising water.

7. A bioactive composition according to claim 6 wherein the bioactive compound, the triazine compound, and the water are substantially uniformly dispersed.

8. A bioactive composition according to claim 7 containing substantially no undissolved bioactive compound.

9. A bioactive composition according to claim 1 wherein the bioactive compound is a drug.

10. A bioactive composition according to claim 1, wherein the triazine compound is zwitterionic.

11. A bioactive composition according to claim 6, comprising a chromonic M or N phase.

12. A bioactive composition according to claim 1 wherein the triazine compound comprises: 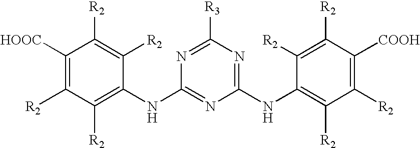
and proton tautomers and salts thereof.

13. A bioactive composition according to claim 12, wherein each R₂ is independently selected from the group consisting of hydrogen, an unsubstituted alkyl group, or an alkyl group substituted with a hydroxy, ether, ester, sulfonate, or halide functional group.

14. A bioactive composition according to claim 12, wherein R₃ comprises a heteroaromatic ring selected from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

15. A bioactive composition according to claim 14, wherein R₃ comprises a heteroaromatic ring derived from pyridine or imidazole.

16. A bioactive composition according to claim 15, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

17. A method for preparing a bioactive composition comprising:
(a) providing a bioactive compound;
(b) providing a triazine compound comprising: 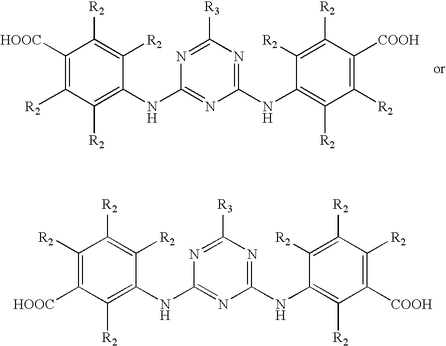
wherein each R₂ is independently selected from any electron donating group, electron withdrawing group and electron neutral group; and
R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃, and proton tautomers and salts thereof; and
(c) combining the bioactive compound, the triazine compound, and a solvent to form a bioactive composition.

18. A method for preparing a bioactive composition according to claim 17, wherein the solvent comprises water.

19. A method for preparing a bioactive composition according to claim 18, wherein the triazine is dissolved in an aqueous solution prior to combining with the bioactive compound.

20. A method for preparing a bioactive composition according to claim 19, wherein the aqueous triazine solution exhibits a chromonic M or N phase.

21. A method for preparing a bioactive composition according to claim 20, wherein the bioactive compound is a drug.

22. A method for increasing the solubility of a bioactive compound in a bioactive composition comprising:
(a) providing a bioactive compound;
(b) providing a triazine compound comprising: 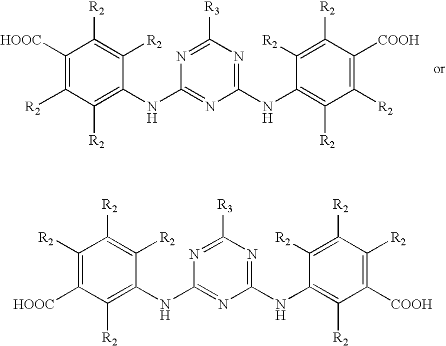
wherein each R₂ is independently selected from any electron donating group, electron withdrawing group and electron neutral group; and
R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃,
and proton tautomers and salts thereof; and
(c) combining the bioactive compound, the triazine compound, and a solvent to form a composition characterized in that the amount of bioactive compound dissolvable in the composition is greater than the amount of bioactive compound dissolvable in the same composition not containing the triazine compound.

23. A method for increasing the solubility of a bioactive compound in a bioactive composition according to claim 22, wherein the ratio of the amount of bioactive compound dissolvable in the dosage form to the amount of bioactive compound dissolvable in the same composition not containing the triazine compound is greater than 2:1.

24. A method for increasing the stability of a bioactive compound in a bioactive composition comprising:
(a) providing a bioactive compound;
(b) providing a triazine compound comprising: 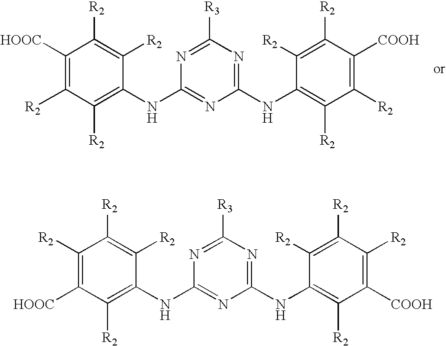
wherein each R₂ is independently selected from any electron donating group, electron withdrawing group and electron neutral group; and
R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃, and proton tautomers and salts thereof; and
(c) combining the bioactive compound, the triazine compound, and a solvent to form a bioactive composition characterized in that the stability of the bioactive compound in the composition is greater than the stability of the bioactive compound in the same composition not containing the triazine compound.

25. A method for increasing the stability of a bioactive compound in a bioactive composition according to claim 24, wherein the stability of the bioactive compound in the composition is characterized by the reduction in amount of bioactive compound over time, and where said reduction in amount of bioactive compound over time is less than the reduction in amount of bioactive compound over time in the same composition not containing the triazine compound.

26. A method for increasing the stability of a bioactive compound in a bioactive composition according to claim 25, wherein the reduction in amount of bioactive compound over time in the composition is measured after storage at conditions of 25° C./60% RH for 3 months.

27. A method for increasing the stability of a bioactive compound in a bioactive composition according to claim 25, wherein the reduction in amount of bioactive compound over time in the composition is measured after storage at conditions of 40° C./75% RH for 3 months.

28. A method for drug delivery comprising:
(a) providing a bioactive composition according to claim 9;
(b) delivering the bioactive composition to an organism; and
(c) allowing the bioactive composition to remain in contact with a portion of the organism for a period of time sufficient to provide a therapeutic effect resulting from delivery of the active agent.

29. A method for drug delivery according to claim 28, wherein the bioactive composition is delivered to an animal orally.

30. A method for drug delivery according to claim 28, wherein the bioactive composition is delivered to an animal by intravenous or intramuscular injection.

31. A bioactive composition comprising:
a bioactive compound, and
a triazine compound comprising: 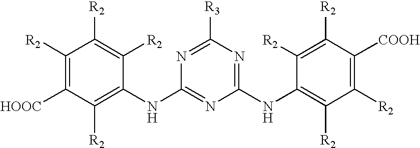
wherein each R₂ is independently selected from any electron donating group, electron withdrawing group and electron neutral group; and
R₃ is selected from the group consisting of substituted heteroaromatic rings, unsubstituted heteroaromatic rings, substituted heterocyclic rings, and unsubstituted heterocyclic rings, that are linked to the triazine group through a nitrogen atom within a ring of R₃,
and proton tautomers and salts thereof.
